(19) United States
(12) Patent Application Publication
(10) Pub. No.: US 2024/0142425 A1
(43) Pub. Date: May 2, 2024

(54) SYSTEM FOR TESTING BANDAGE WITH ODOR INHIBITION

(71) Applicant: Biodaptive Advanced Materials, LLC, Ronkonkoma, NY (US)

(72) Inventor: Matthew Robert HENRY, State College, PA (US)

(21) Appl. No.: 18/386,444

(22) Filed: Nov. 2, 2023

Related U.S. Application Data
(60) Provisional application No. 63/421,823, filed on Nov. 2, 2022.

(51) Int. Cl. G01N 33/00

(52) U.S. Cl. CPC G01N 33/0054

(57) ABSTRACT

A system for determining efficacy of an odor absorbing bandage includes a sample chamber in which an odor absorbing bandage or other article is received, where the sample chamber is in fluid communication with an ammonia detector that allows for a determination of an amount of ammonia that escapes the sample chamber because it is not absorbed by the odor absorbing bandage.

### CROSS-REFERENCE TO RELATED APPLICATIONS

The present application claims benefit of and priority to U.S. Provisional Patent Application No. 63/421,823 entitled SYSTEM FOR TESTING BANDAGE WITH ODOR INHIBITION filed Nov. 2, 2022, the entire content of which is incorporated by reference herein.

### BACKGROUND

### Field of the Disclosure

The present invention relates to a system for testing odor inhibition in a bandage with odor inhibiting material.

### Related Art

An offensive odor coming from a wound site may be an indication of bacterial infection or necrotic tissue. Such offensive orders are not in themselves harmful, however, they are unpleasant and distracting to the patient and caregivers. In addition, offensive odors may be embarrassing to the patient and/or family members. A variety of bandages have been developed to mitigate such odors. These bandages typically include material that will absorb or trap volatile compounds that are responsible for offensive odors such as activated charcoal, cyclodetrin, or PHMB, to name a few. One problem associated with such odor absorbing bandages is the lack of a reliable test to assess the effectiveness of these bandages. Conventionally, the effectiveness of such odor controlling bandages was based on clinical tests in which healthcare workers would submit subjective assessments as to whether odor had improved. There was no standardized objective test available to assess the effectiveness of such bandages.

Accordingly, it would be beneficial to provide a system and method that provides an objective indication of the effectiveness of odor absorbing bandages or other odor absorbing materials to avoid these and other problems.

### SUMMARY

It is an object of the present disclosure to provide a system and method for providing an objective indication of the effectiveness of an odor reducing bandage.

A system for determining efficacy of an odor absorbing bandage includes: a sample chamber configured to receive the odor absorbing bandage; and an ammonia detector in fluid communication with the sample chamber and configured to detect an amount of ammonia emanating from the sample chamber.

In embodiments, the sample chamber includes: a base; and a removable top connected to the base and including a first opening formed therein to provide fluid communication between the sample chamber and the ammonia detector.

In embodiments, the sample chamber includes a seal preventing release of vapor between the removable top and the base.

In embodiments, the system includes an adapter element connected to the first opening formed in the removable lid and providing fluid communication between the sample chamber and the ammonia detector.

In embodiments, the system includes an adaptor element connecting an interior of the sample chamber to the ammonia detector such that ammonia vapor in the sample chamber passes through the adaptor element and enters the ammonia detector.

In embodiments, the ammonia detector is a Draeger tube.

In embodiments, the ammonia detector collects an air sample from the sample chamber.

In embodiments, the air sample is analyzed using gas chromatography to determine a level of ammonia in the air sample.

In embodiments, the air sample is analyzed using a flame photometric detector to determine a level of ammonia in the air sample.

In embodiments, the system includes a gas chromatography device in fluid communication with the ammonia detector to detect ammonia present in the sample chamber.

A system for determining efficacy of an odor absorbing bandage includes an ammonia chamber configured to receive an ammonia sample; a sample chamber mounted on the ammonia chamber and configured to receive the odor absorbing bandage, wherein the sample chamber is in fluid communication with the ammonia chamber such that ammonia vapors pass from the ammonia chamber into the sample chamber through the odor absorbing bandage; and an ammonia detector in fluid communication with the sample chamber and configured to detect an amount of ammonia emanating from the sample chamber.

In embodiments, the ammonia chamber includes a first top element including at least a first opening configured to allow ammonia vapors to pass therethrough.

In embodiments, the sample chamber includes: a base including a second opening formed in a bottom surface thereof and in fluid communication with the second first opening in the first top element of the ammonia chamber such that ammonia vapors flow into the sample chamber from the ammonia chamber; and a removable top connected to the base and including a third opening formed in a top surface thereof to provide fluid communication between the sample chamber and the ammonia detector.

In embodiments, the sample chamber includes a seal preventing release of vapor between the removable lid and the base.

In embodiments, the system includes an adapter element connected to the third opening formed in the removable top and providing fluid communication between the sample chamber and the ammonia detector.

In embodiments, the system includes an adaptor element connecting an interior of the sample chamber to the ammonia detector such that ammonia vapor in the sample chamber passes through the adaptor element and enters the ammonia detector.

In embodiments, the ammonia detector is a Draeger tube.

In embodiments, the ammonia detector collects an air sample from the sample chamber.

In embodiments, the air sample is analyzed using gas chromatography to determine a level of ammonia in the air sample.

In embodiments, the air sample is analyzed using a flame photometric detector to determine a level of ammonia in the air sample.

In embodiments, the system includes a gas chromatography device in fluid communication with the ammonia detector to detect ammonia present in the sample chamber.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates a system for measuring effectiveness of an odor absorbing bandage in accordance with an exemplary embodiment of the present disclosure;

FIG. 2 illustrates a more detailed view of a top of a sample chamber used in the system of FIG. 1 in accordance with an embodiment of the present disclosure;

FIG. 3 illustrates an exemplary odor absorbing bandage that may be tested in the system of FIG. 1;

FIG. 3A illustrates an exploded view of the exemplary odor absorbing bandage of FIG. 3;

FIG. 4 illustrates a more detailed view of a base of the sample chamber used in the system of FIG. 1 in accordance with an embodiment of the present disclosure;

FIG. 5 illustrates a system for measuring effectiveness of an odor absorbing bandage in accordance with another embodiment of the present disclosure;

FIG. 6 illustrates a detailed view of an ammonia detector suitable for use in the systems of FIG. 1 and FIG. 5;

FIG. 7 illustrates a more detailed view of a top of a sample chamber used in the system of FIG. 5 in accordance with an embodiment of the present disclosure;

FIG. 8 illustrates a more detailed view of a base of the sample chamber and a top of an ammonia chamber used in the system of FIG. 5 in accordance with an embodiment of the present disclosure;

FIG. 9 illustrates a bottom of the ammonia chamber used in the system of FIG. 5 in accordance with an embodiments of the present disclosure;

FIG. 10 illustrates an example of an ammonia sample that may be provided in the ammonia chamber in the system of FIG. 5 in accordance with an embodiment of the present disclosure; and

FIG. 11 is a more detailed view of an exemplary bandage that may be placed in the sample chamber of the system of FIG. 5 in accordance with an embodiment of the present disclosure.

### DETAILED DESCRIPTION OF THE EXEMPLARY EMBODIMENTS

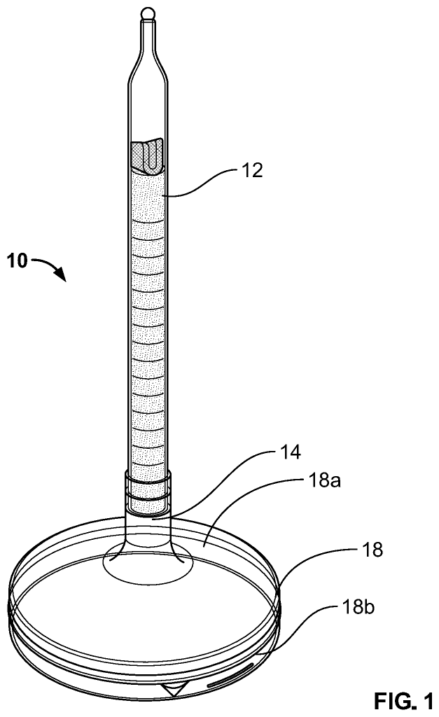

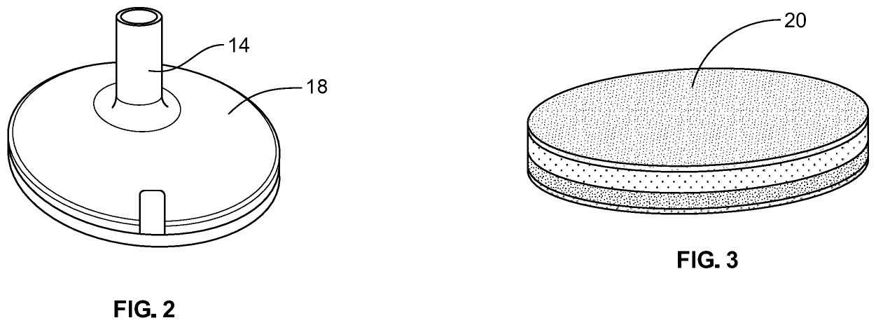

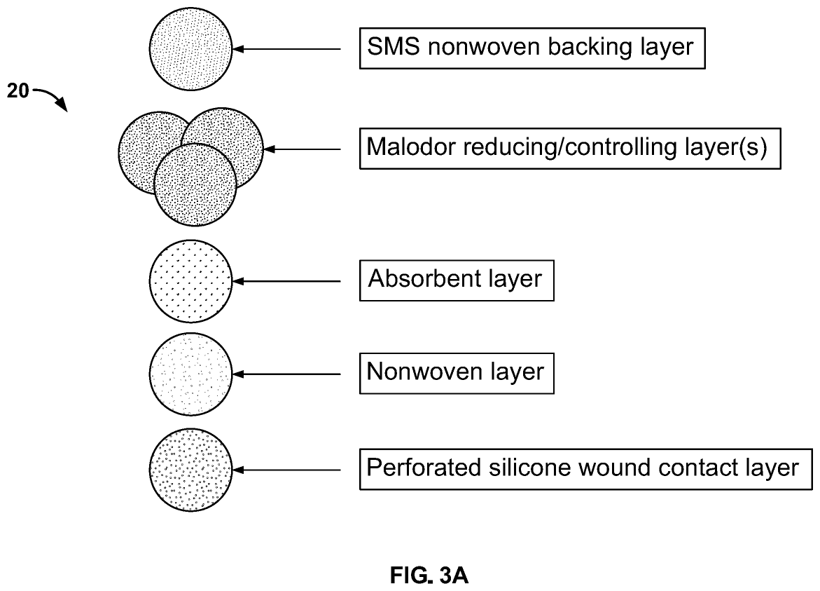

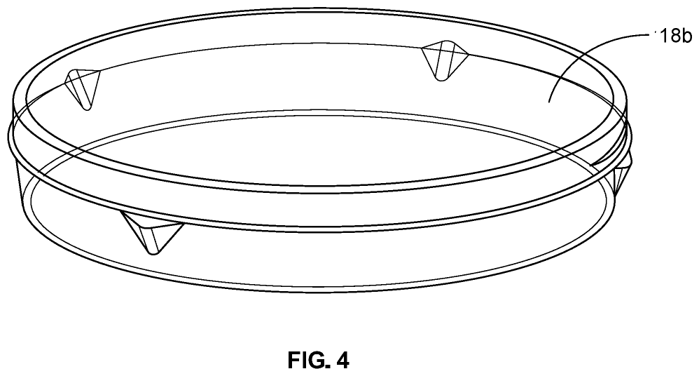

A system 10 for determining effectiveness of an odor absorbing bandage 20 (see FIG. 3) in accordance with an embodiment of the present disclosure is illustrated in FIG. 1. In embodiments, the system 10 includes an ammonia detector 12 that may be used to detect ammonia that has passed through the bandage being tested. In embodiments, the ammonia detector 12 may provide an indication of the presence of ammonia and may also provide an indication of an amount of ammonia that is present. Ammonia is one of the primary volatile substances that may be produced in an infected or necrotic wound such that measuring the discharge of ammonia through an odor absorbing bandage provides a quantitative indication of the effectiveness of the bandage in absorbing odor. In embodiments, the system 10 may detect other substances that cause odor and the ammonia detector 12 may be configured to detect the presence of these other substances. In embodiments, odors may be caused by volatile organic compounds (VOCs) that are produced by microorganisms. In embodiments, these volatile organic compounds may include dimethyl trisulfide, acetic acid, isobutyric acid, butyric acid, and isovaleric acid, to name a few. In embodiments, the ammonia detector 12 may be configured to detect one of these volatile organic compounds. In FIG. 1, the ammonia detector 12 is a Draeger tube. In embodiments, other ammonia detectors may be used. In embodiments, the ammonia detector 12 may be used to collect an air sample of air from the sample chamber 18. In embodiments, the air sample may be withdrawn from the detection element 12. In embodiments, the air sample may be analyzed using gas chromatography. In embodiments, gas chromatography may be used in conjunction with a mass spectrometer to detect ammonia, or other volatile organic substances in the air sample. In embodiments, gas chromatography may be used in conjunction with a flame photometric detector to detect the presence of volatile sulfur compounds in the air sample. In embodiments, the ammonia detector 12 may use an electronic nose device to identify volatile organic substances in the air sample. In embodiments, the system 10 may be directly connected to gas chromatography equipment, for example, via a tube or other connector from the detection element 12 which may be used to provide the air sample to the gas chromatography equipment to identify the presence of volatile organic substances.

In embodiments, the ammonia detector 12 may be in fluid communication with an adapter element 14 that is secured to or integrated with a top 18a of a sample chamber 18. In embodiments, the top 18a may be a removable lid that is removably connected to a base 18b. In embodiments, as noted above, the ammonia detector 12 may be directly connected to or otherwise in fluid communication with the chamber 18 without using the adaptor element 14. In embodiments, the adapter element 14 provides fluid communication between the sample chamber 18 and the ammonia detector 12 to allow volatile substances like ammonia to pass from the sample chamber to the ammonia detector. In embodiments, the top 18a of the sample chamber 18 includes an opening that is mated with the adapter element 14 to allow volatile substances from the sample chamber 18 to flow into the detecting element 12. In embodiments the adaptor element 14 may be integrated into the top 18a. In embodiments, the sample chamber 18 may be a petri dish and the top 18a may be a removable lid with an opening provided in the top thereof for mating with the adapter 14. In embodiments, the top 18a may be adapted to connect directly to the ammonia detector 12 in which case the adapter 14 may not be necessary.

In embodiments, the bandage 20 to be tested is placed in the sample chamber 18 for testing. FIG. 3 illustrates an exemplary mock bandage including odor control. As illustrated in FIG. 3A, in embodiments, the bandage 20 may include an SMS nonwoven backing layer on top with one or more malodor reducing layers provided under the SMS nonwoven backing layer. As noted above, these malodor reducing layers may include activated charcoal or PHMB, to name a few. An absorbent layer may be provided below the malodor reducing layers and a nonwoven layer may be provided below that. A perforated silicone wound contact layer is provided on the bottom of the bandage 20 and will be the layer in contact with the user's skin. In embodiments, a solution that includes nutrients, urea and a known concentration of bacteria will be applied to a bottom surface of the silicone layer. After the solution is fully absorbed, the bandage 20 is placed in the sample chamber 18 and the top 18a, is placed on top of the base 18b. The ammonia detector 12 is connected to the adapter 14 which is connected to the cover or lid 18a. A seal may be provided between the cover 18a and the base 18b of the sample chamber to ensure that all volatile substances exit through the adapter 14 and into detecting element 12. While a bandage 20 is illustrated in the present application, the system 10 may be used to test the odor absorbing effectiveness of other items such as textiles, foam and wound dressings, to name a few.

In embodiments, the device 10 with the sample 20 is incubated at 36 degrees C., or another temperature suitable for use with the bandage 20 or other material being tested. Over time, the bacteria on the silicone layer will convert the urea and nutrients into ammonia which will pass through the layers of the bandage 20. Any ammonia that is not absorbed by the various layers of the bandage will pass through the adapter 14 and into the ammonia detector 12. In embodiments, the device 10 is observed hourly during incubation to determine ammonia production as indicated by the ammonia detector 12. In embodiments, observations may be made more frequently or less frequently. After a predetermined period of time, which may be based on saturation of the ammonia detector 12, the bandage 20 may be removed from the sample chamber 18 and the population of bacteria may be determined and compared to the amount originally provided to the bandage. In embodiments, such a comparison will determine whether the bandage 20 had an anti-microbial effect, which would have affected the generation of ammonia as well as the amount of ammonia that was not absorbed and was detected in the ammonia detector 12. Based on the amount of ammonia detected by the ammonia detector 12, a determination can be made as to the effectiveness of the bandage 20 in absorbing odors. By recording the bacteria count before and after testing, it can be confirmed that the reduction of ammonia detected is based on absorption of ammonia by the bandage 20 rather than reduction in the number of bacteria producing the ammonia.

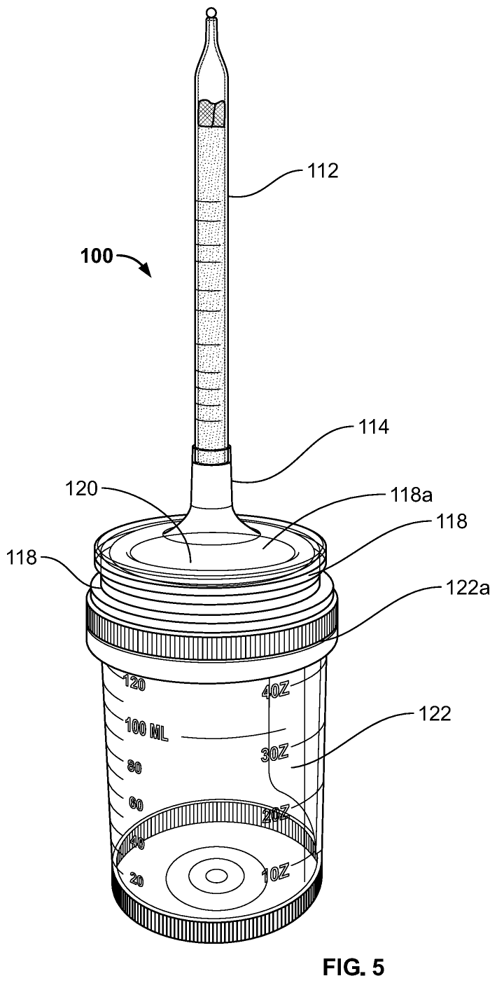

FIG. 5 illustrates another system 100 in accordance with an embodiment of the present disclosure. In embodiments, the system 100 includes an ammonia detector 112 which is connected via adapter 114 to a sample chamber 118. In embodiments, the ammonia detector 112 and the adapter 114 are similar in structure and function to the ammonia detector 12 and adapter 14 discussed above. FIG. 6 shows a more detailed view of the ammonia detector 112 which may be used in the system 10 as the ammonia detector 12 as well.

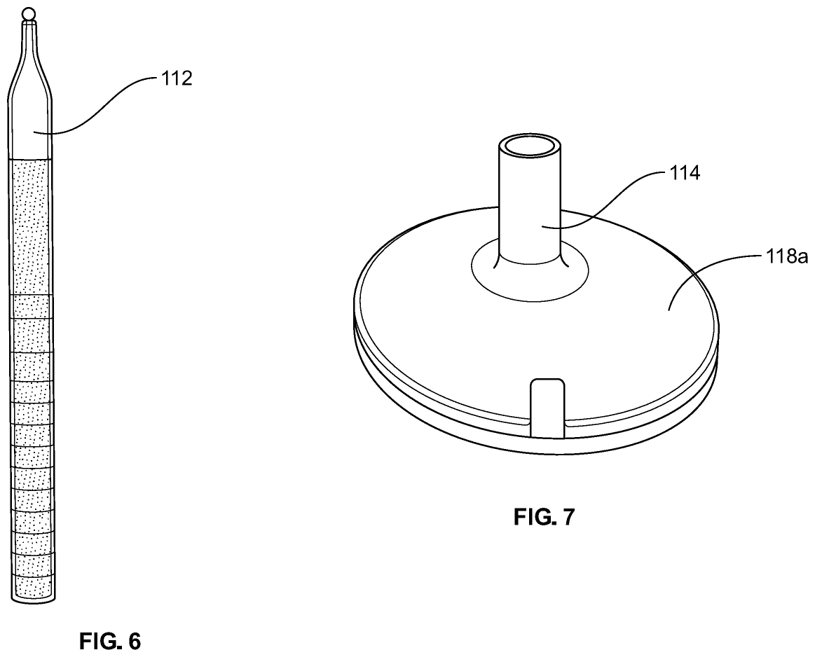

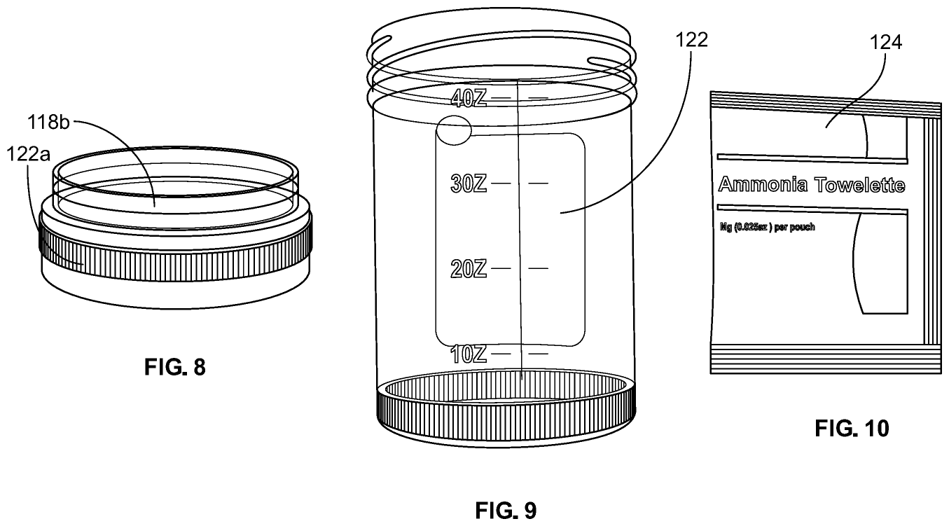

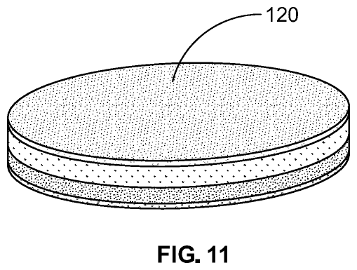

The top 118a of the sample chamber 118 is similar in structure and function to the top 18a of the sample chamber 18 discussed above as can be seen in FIG. 7, for example. The base or bottom 118b of the sample chamber 118 may be secured to a top 122a of an ammonia chamber 122. In embodiments, a bottom surface of the base 118b of the sample chamber 118 includes an opening or openings that are in fluid communication with a corresponding opening or openings formed through the top 122a of the ammonia chamber 122. In embodiments, the ammonia chamber 122 receives an ammonia sample 124 (see FIG. 10, for example), which may be an ammonia impregnated wipe of the type illustrated in FIG. 10 or any other suitable sample and the openings in the top 122a and the bottom surface of the base 118b allow ammonia vapors to enter the sample chamber 118. In embodiments, a bandage 120 (FIG. 11) to be tested may be positioned over the openings in the bottom surface of the base 118b such that ammonia vapors pass through the bandage 120.

In embodiments, the ammonia source 124 may be unwrapped or otherwise allowed to release ammonia vapors in the ammonia chamber 124. The vapors pass through the openings in the top 122a and the base 118b and through the bandage 120. Any vapors that are not absorbed in the bandage 120 pass through the adapter and into the detecting element 112. The system 100 is observed for a period of time, which again, may be based on a saturation point of the detection element 112 and ammonia levels may be recorded. Based on the recorded ammonia levels, a determination can be made as to the efficacy of the bandage 120 in absorbing odors (the ammonia). The system 100 does not require the use of bacteria or the nutrients and urea needed to feed them such that it is arguably simpler to use. Further, since no bacteria is used, the system 100 may be safer to operate as there is no concern with respect to contamination.

Both of the systems 10, 100 provide for objective measurements of the efficacy of the sample bandages in absorbing odors such that both systems allow for objective testing of odor absorbing bandages.

Although the present invention has been described in relation to particular embodiments thereof, many other variations and modifications and other uses will become apparent to those skilled in the art. It is preferred, therefore, that the present invention be limited not by the specific disclosure herein.

What is claimed is:

1. A system for determining efficacy of an odor absorbing bandage comprising:
a sample chamber configured to receive the odor absorbing bandage; and
an ammonia detector in fluid communication with the sample chamber and configured to detect an amount of ammonia emanating from the sample chamber.

2. The system of claim 1, wherein the sample chamber includes:
a base; and
a removable top connected to the base and including a first opening formed therein to provide fluid communication between the sample chamber and the ammonia detector.

3. The system of claim 2, wherein the sample chamber further comprises a seal preventing release of vapor between the removable top and the base.

4. The system of claim 2, further comprising an adapter element connected to the first opening formed in the removable lid and providing fluid communication between the sample chamber and the ammonia detector.

5. The system of claim 1, further comprising an adaptor element connecting an interior of the sample chamber to the ammonia detector such that ammonia vapor in the sample chamber passes through the adaptor element and enters the ammonia detector.

6. The system of claim 1, wherein the ammonia detector is a Draeger tube.

7. The system of claim 1, wherein the ammonia detector collects an air sample from the sample chamber.

8. The system of claim 7, wherein the air sample is analyzed using gas chromatography to determine a level of ammonia in the air sample.

9. The system of claim 7, wherein the air sample is analyzed using a flame photometric detector to determine a level of ammonia in the air sample.

10. The system of claim 1, further comprising a gas chromatography device in fluid communication with the ammonia detector to detect ammonia present in the sample chamber.

11. A system for determining efficacy of an odor absorbing bandage comprising:
an ammonia chamber configured to receive an ammonia sample;
a sample chamber mounted on the ammonia chamber and configured to receive the odor absorbing bandage, wherein the sample chamber is in fluid communication with the ammonia chamber such that ammonia vapors pass from the ammonia chamber into the sample chamber through the odor absorbing bandage; and
an ammonia detector in fluid communication with the sample chamber and configured to detect an amount of ammonia emanating from the sample chamber.

12. The system of claim 11, wherein the ammonia chamber includes a first top element including at least a first opening configured to allow ammonia vapors to pass therethrough.

13. The system of claim 12, wherein the sample chamber includes:
a base including a second opening formed in a bottom surface thereof and in fluid communication with the first opening in the first top element of the ammonia chamber such that ammonia vapors flow into the sample chamber from the ammonia chamber; and
a removable top connected to the base and including a third opening formed in a top surface thereof to provide fluid communication between the sample chamber and the ammonia detector.

14. The system of claim 13, wherein the sample chamber further comprises a seal preventing release of vapor between the removable lid and the bade.

15. The system of claim 13, further comprising an adapter element connected to the third opening formed in the removable top and providing fluid communication between the sample chamber and the ammonia detector.

16. The system of claim 11, further comprising an adaptor element connecting an interior of the sample chamber to the ammonia detector such that ammonia vapor in the sample chamber passes through the adaptor element and enters the ammonia detector.

17. The system of claim 11, wherein the ammonia detector is a Draeger tube.

18. The system of claim 11, wherein the ammonia detector collects an air sample from the sample chamber.

19. The system of claim 18, wherein the air sample is analyzed using gas chromatography to determine a level of ammonia in the air sample.

20. The system of claim 18, wherein the air sample is analyzed using a flame photometric detector to determine a level of ammonia in the air sample.

21. The system of claim 11, further comprising a gas chromatography device in fluid communication with the ammonia detector to detect ammonia present in the sample chamber.
